SEQUENCE LISTING (13 sequences)

SEQ ID NO: 1 - DNA, 30 bp, Artificial Sequence
  Description of Artificial Sequence PCR primers for A. thaliana for CMT2.
catggtttgtggaggacctccntgycargg

SEQ ID NO: 2 - DNA, 32 bp, Artificial Sequence
  Description of Artificial Sequence PCR primers for A. thaliana for CMT2.
ttgcatcattccgaatctacaytgrtanyyca

SEQ ID NO: 3 - DNA, 32 bp, Artificial Sequence
  Description of Artificial Sequence PCR primers for A. thaliana CMT3.
gttagagagtgtgctagacttcarggnttycc

SEQ ID NO: 4 - DNA, 31 bp, Artificial Sequence
  Description of Artificial Sequence PCR primers for A. thaliana CMT3.
caacaggaacagcaacagcrttnccnayytg

SEQ ID NO: 5 - DNA, 22 bp, Artificial Sequence
  Description of Artificial Sequence  RT-PCR primers for CMT2.
gtctttggtgggatgaaactgt

SEQ ID NO: 6 - DNA, 24 bp, Artificial Sequence
  Description of Artificial Sequence  RT-PCR primers for CMT2.
cttgaagctgagggtaagttgaat

SEQ ID NO: 7 - DNA, 23 bp, Artificial Sequence
  Description of Artificial Sequence RT-PCR primers for CMT3.
gtaaaagcttgcagcataaccac

SEQ ID NO: 8 - DNA, 23 bp, Artificial Sequence
  Description of Artificial Sequence RT-PCR primers for CMT3.
taactttttagggactccgaagg

SEQ ID NO: 9 - DNA, 25 bp, Artificial Sequence
  Description of Artificial Sequence  PCR primer for hda1 gene-specific amplification.
ggtaatggatactggcggcaattcg

SEQ ID NO: 10 - DNA, 25 bp, Artificial Sequence
  Description of Artificial Sequence PCR reverse primer for hda1 gene-specific amplification.
accacccaagagcagtaggggaaca

SEQ ID NO: 11 - DNA, 520 bp, Arabidopsis thaliana
attgtttatatgttgttgatgtttagggaggtgttgatgttgtctgcggggggccaccatgccaaggaatcagtggtcacaaccgcttcaggaacttattggaccctctagaagatcagaaaaacaagcagcttttggtgtatatgaacattgtagaatatttgaagcctaagttcgttttgatggaaaacgtcgttgacatgctgaagatggctaagggctatcttgcacggtttgctgttggacgccttctacagatgaattaccaagtgaggaatggaatgatggcagctggagcttatgggcttgctcagtttcgtttgaggttctttctatggggtgcactccctagtgaggtaaaattcttaatgttttctctttgttttatattaatgaatagatgctgttcttaatttgtatctttctgtttcagataattccgcagttcccacttccaacacatgatttagttcatagaggaaatattgtcaaggagtttcaggtaaactggtatattagt

SEQ ID NO: 12 - protein, 110 aa, Artificial Sequence
  Description of Artificial Sequence Encoded amino acid variations in CMT3B fragment following EMS mutagenesis.
XXXXXXXXXXXXQXIXXXNXFXNLLXXLXXQKNKQXLXYXNIXXYLKXKFXLXXNXXXXLKXXKXYXXXFXXXXXLQXNYQXXNXXXXXXXYXXXQFXLXFFLWXXXXXX

SEQ ID NO: 13 - protein, 23 aa, Artificial Sequence
  Description of Artificial Sequence Encoded amino acid variations in CMT3B fragment following EMS mutagenesis.
IIXQFXXXXXXLXXXXNIXKXFQ

(19) United States
(12) Patent Application Publication
(10) Pub. No.: US 2004/0053236 A1
(43) Pub. Date: Mar. 18, 2004

(54) Reverse genetic strategy for identifying functional mutations in genes of known sequences

(76) Inventors: Claire M. McCallum, Seattle, WA (US); Steven Henikoff, Seattle, WA (US); Trenton Colbert, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/240,456

(22) PCT Filed: Mar. 30, 2001
(86) PCT No.: PCT/US01/10545

(51) Int. Cl. C12Q 1/68

(52) U.S. Cl. 435/6

(57) ABSTRACT

The present invention provides a simple and general reverse genetic strategy for use in organisms where well-developed genetic tools are lacking. In particular, the method comprises contacting an organism or cell with a mutagen which primarily induces point mutations in the genomic DNA; preparing DNA from the organism or cell; amplification of a genomic region of interest; and screening for changes in the mutagenized DNA sequence in the genomic region of interest as compared to the parent organism or cell. Screening for mutations can comprise detection of heteroduplexes to identify a mutant organism or cell. The methods of the present invention are particularly useful for identifying functional mutations in plants.

### STATEMENT AS TO RIGHTS TO INVENTIONS MADE UNDER FEDERALLY SPONSORED RESEARCH AND DEVELOPMENT

This work was supported by grants GM29009 from the National Institutes of Health and National Science Foundation grant DBI 0077737. The U.S. Government may have certain rights in the invention.

### BACKGROUND OF THE INVENTION

One of the most important breakthroughs in the history of genetics was the discovery that mutations can be induced (Muller, J. Genet. 22:299-334 (1930); Stadler, P. VL C. G. 1:274-294(1932)). The high frequency with which ionizing radiation and certain chemicals can cause genes to mutate made it possible to perform genetic studies that were not feasible when only spontaneous mutations were available. As a result, much of our understanding of genetics of higher organisms is based upon studies utilizing induced mutations for analyzing gene function. Alkylating agents, which yield predominantly point mutations, have been especially valuable, since the resulting altered and truncated protein products help to precisely map gene and protein function. Because of the high mutational density and the great utility of point mutations, traditional chemical mutagenesis methods have continued to be popular in phenotypic screens despite the development of other mutagenic tools, such as transposon mobilization (Bingham et al., Cell 25:693-704 (1981)).

With the recent expansion of sequence databanks, locus-to-phenotype reverse genetic strategies have become an increasingly popular alternative to phenotypic screens for functional analysis. Sequence information alone may be sufficient to consider a gene to be of interest, because sequence comparison tools that detect protein sequence similarity to previously studied genes often allow a related function to be inferred. Hypotheses concerning gene function that are generated in this way must be confirmed empirically. Experimental determination of gene function is desirable in other situations as well, for example when a genetic interval has been associated with a phenotype of interest. In such cases, the functions of genes in an interval can be inferred from the phenotypes of induced mutations. Furthermore, the dissection of gene interactions often requires the availability of a range of allele types.

However, most available methods for inferring function rely on techniques that produce a limited range of mutations, are labor intensive, unreliable, or are limited to species in which special genetic tools have been developed. Just as the discovery of induced mutations led to forward genetics, the introduction of rapid reverse genetic methods can have great impact. Routine reverse genetics (Scherer et al., Proc. Natl. Acad. Sci. USA 76:4951-4955 (1979)) has been an important factor in the popularity of baker's yeast over the past two decades, and the RNAI technique (Fire et al., Nature 391:806-811 (1998)) now provides C. elegans investigators with a routine knockout method that has enjoyed huge popularity (Sharp, Genes and Dev. 13:139-141 (1999); Liu et al., Genome Res. 9:859-867 (1999)). In most other eukaryotes however, the situation remains unsatisfactory.

In plants, the two most common methods for producing reduction-of-function mutations are antisense RNA suppression (Schuch, Mutat. Res. 211:231-241 (1989); de Lange et al., Curr. Top. Microbiol. ImmunoL 197:57-75 (1995); Hamilton et al., Curr. Top. Microbiol. Immunol. 197:77-89 (1995); Finnegan et al., Proc. Natl. Acad. Sci. USA 93:8449-8454 (1996)) and insertional mutagenesis (Altmann et al., Mol Gen. Genet. 247:646-652 (1995); Smith et al., Plant J. 10:721-732 (1996); Azpiroz-Leehan, et al., Trends Genet. 13:152-156 (1997); Long et al., Methods Mol. Biol. 82:315-328 (1998); Martienssen, R. A. Proc. Natl. Acad Sci. USA 95:2021-2026 (1998); Pereira et al., Methods Mol. Biol. 82:329-338, (1998); van Houwelingen et al., Plant J. 13: 39-50 (1998); Speulman et al., Plant Cell 11:1853-1866 (1999)). However, antisense RNA suppression requires considerable effort for any given target gene before knowing whether it will work at all, and insertional mutagenesis occurs at a low frequency per genome.

There is current interest in RNAi-related suppression (Waterhouse et al., Proc. Natl. Acad. Sci. USA 95:13959-13964 (1998); Baulcombe, Arch Virol. Suppl. 15:189-201 (1999)), however, its efficacy is not yet clear. Because these techniques rely either on Agrobacterium T-DNA vectors for transmission or on an endogenous tagging system, their usefulness as general reverse genetics methods is limited to very few plant species, for which this vector works. Moreover, these techniques produce a very limited range of allele types. Therefore, as the amount of DNA sequence data grows for Arabidopsis and other organisms, it is important to develop genome-scale reverse genetic strategies that are automated, broadly applicable and capable of creating the wide range of mutant alleles that is needed for functional analysis.

The present invention provides a reverse genetic strategy that combines the high density of mutations offered by traditional mutagenesis methods with rapid mutational screening to discover induced lesions. The method, designated TILLING (Targeting Induced Local Lesions In Genomes), combines the efficiency of mutagenesis methods, e.g., chemical-induced (for example, using ethyl methanesulfonate (EMS)(Koornneef et al., Mutat. Res. 93:109-123 (1982))), or radiation with the ability of mutation analysis tools, such as the detection of single base pair changes by heteroduplex analysis (Underhill et al., Genome Res. 7:996-1005 (1997)) to identify, concurrent with screening, the locating of the mutation thus eliminating needless follow-up in areas such as introns, and non-conserved sequences. The TILLING method generates a wide range of mutant alleles, is fast and automatable, and is applicable to any organism that can be mutagenized, stored and propagated.

### SUMMARY OF THE INVENTION

The present invention provides a reverse genetic method for identifying functional mutations in a gene of known sequence comprising treating an organism or cell with mutagen which induces mutations in the DNA of an organism or cell; preparing isolated genomic DNA from the mutagenized organism or cell; amplifying a region of a gene of known sequence; and screening for mutations in the mutagenized DNA sequence in the gene as compared to the same sequence of the gene in the wild type parent organism or cell. The method designated TILLING, for Targeted Induced Local Lesion in Genomes, combines the high density of mutations provided by traditional mutagenesis methods with rapid mutational analysis methods to identify mutations of interest in genes of known sequence without inserting heterologous nucleic acids into an organism or cell.

Methods for mutagenizing the genome of an organism or a cell to induce mutations can include treating an organism or cell with chemical agents or radiation. In particular, a traditional chemical mutagen such as ethyl methanesulfonate, methylmethane sulfonate, N-ethyl-N-nitrosourea, triethyhnelamine, diepoxyalkanes (diepoxyoctane, diepoxybutane, and the like), 2-methoxy-6-chloro-9[3-(ethyl-2-chloro-ethyl) aminopropylamino] acridine dihydrochloride, formaldehyde, and the like can be used. Samples, of the mutagenized organism or cells are then collected and DNA samples are pooled for analysis by nucleic acid amplification, heteroduplex analysis to determine the approximate location of the mutation, and optionally DNA sequencing.

Nucleic acid amplification methods suitable for use in the methods of the present invention include, but are not limited to PCR methods such as RT-PCR. Primers are selected to amplify a region of the genome comprising the gene of interest. The PCR product is then analyzed for the presence of mutations. Mutations can be detected, for example, by single-stranded conformational polymorphism or by heteroduplex analysis, and the like. Methods for heteroduplex analysis compatible with the methods of the present invention include constant denaturant capillary electrophoresis, denaturing high pressure liquid chromatography, and enzyme or chemical digestion of nucleotide mismatches followed by separation and detection of the digested DNA. Each of these methods have been used previously to identify naturally occurring polymorphisms consisting of single base changes in genes of interest (Cotton et al., Mutation Detection: A Practical Approach, IRL Press, Oxford, England (1998)). In TILLING the mutations detected are either missense or nonsense mutations which result in altered or truncated protein products. The organisms or cells analyzed by the disclosed methods can be either homozygous or heterozygous for the mutation of interest.

The methods of the present invention are applicable to any organism which can be heavily mutagenized, including both plants and animals. In a specific embodiment, TILLING has been applied to two Arabidopsis thaliana chromomethylase genes related to CMT 1, a DNA methyltransferase homologue with a chromodomain (Henikoff and Comai, Genetics 149:307-318 (1998)). The methods are also applicable to other plants, particularly crop plants such as maize, alfalfa, wheat, barley, soy beans, cotton, pine, rice, legumes, i.e., Medicago truncatula, and the like. Using the methods of the present invention it is possible to select for plants with phenotypic variations of commercial interest without introducing foreign DNA of any type into the plant genome.

BRIEF DESCRIPTION OF THE DRAWINGS

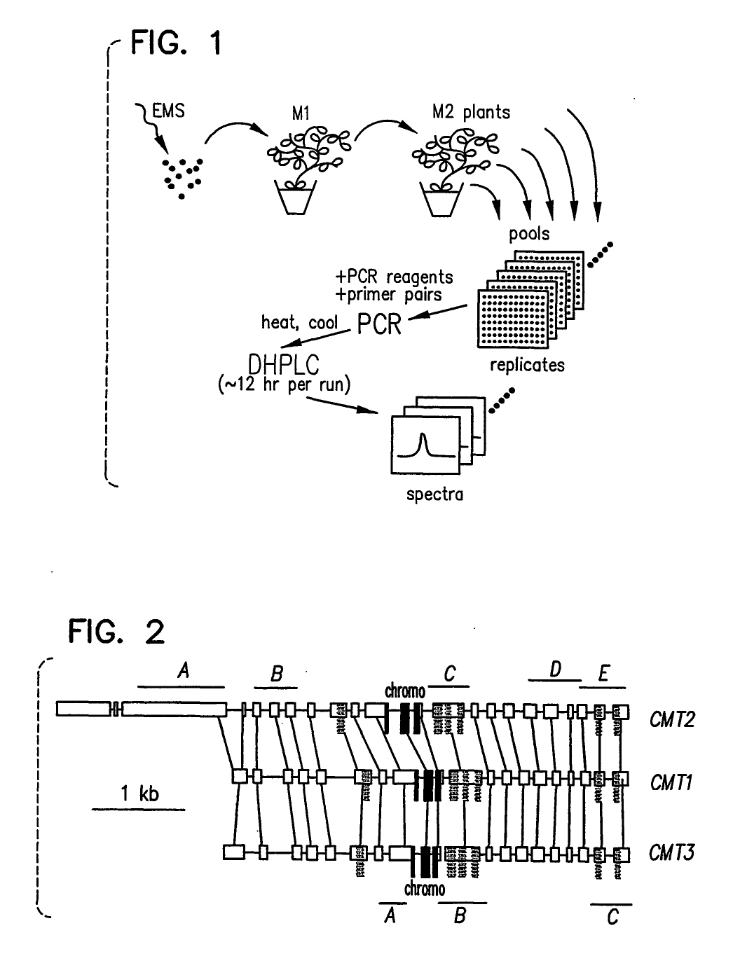

FIG. 1 depicts in the form of a cartoon the TILLING strategy applied to a plant such as Arabidopsis thaliana.

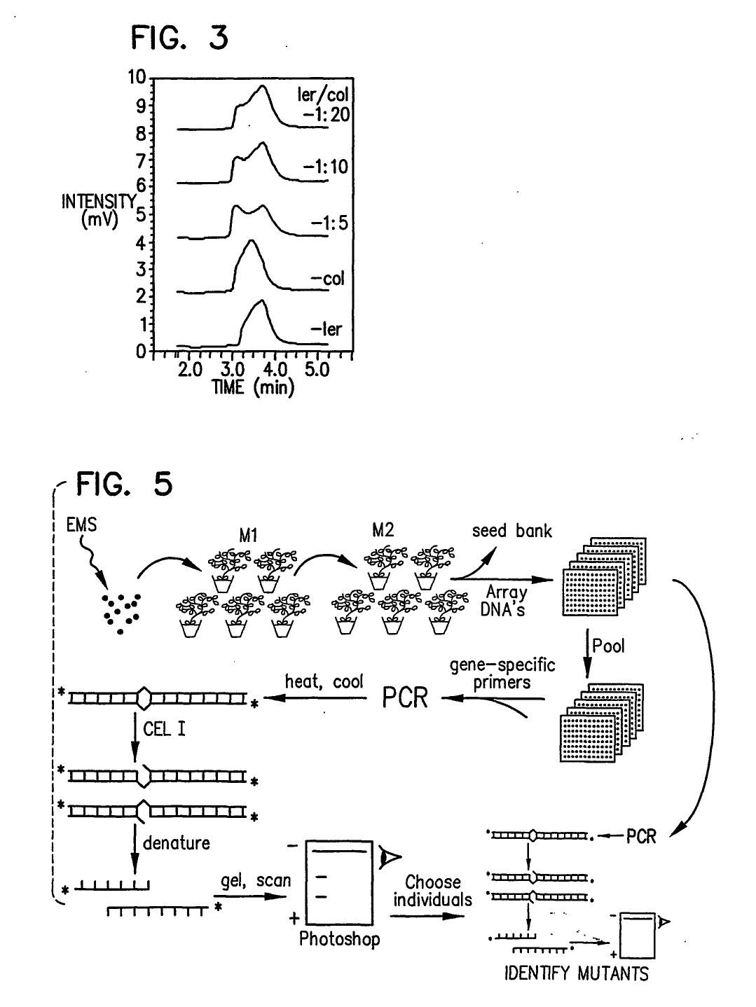

FIG. 2 depicts the structure of Arabidopsis thaliana chromomethylase genes. Exons are shown as boxes with cytosine DNA methyltransferase blocks (black rectangles) and chromodomain blocks (gray rectangles) indicated. Fragments used for TILLING analysis are indicated as horizontal lines above CMT2 and below CMT3.

FIG. 3 provides a depiction of dHPLC chromatograms showing typical sensitivity for detection of a transition mutation on a PCR fragment, where Ler and Col templates, which differ by a single CIG to T/A change, have been mixed in the indicated ratios and amplified. Retention time on the dHPLC column is plotted against intensity of the signal in millivolts (mV).

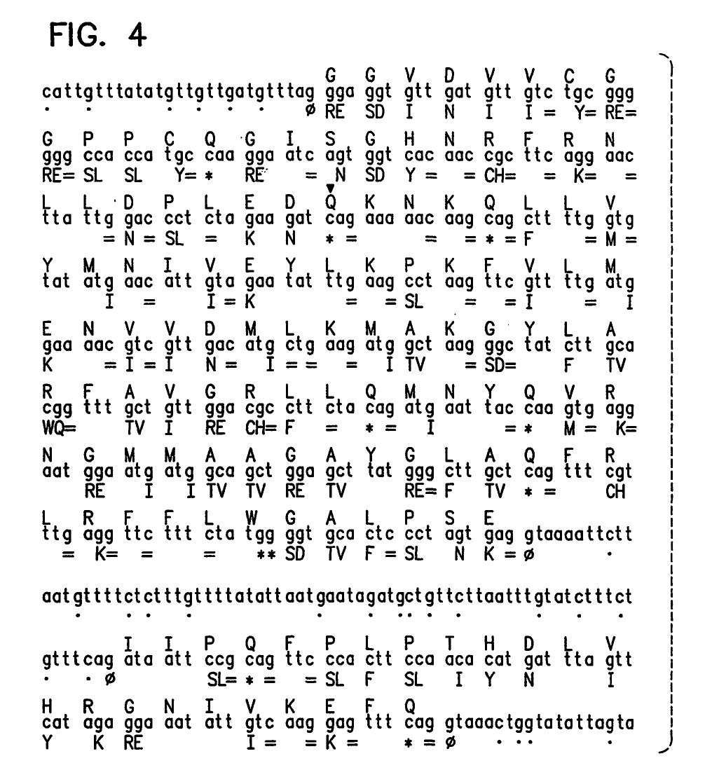

FIG. 4 depicts the sites that are most susceptible to base transition mutations after treatment with EMS for the CMT3B fragment (the nucleotide sequence of the fragment is depicted as SEQ ID NO: 11, and the amino acids encoded by the wild-type sequence and the mutations detected for each amino acid position for this fragment are depicted as SEQ ID NO: 12 and SEQ ID NO: 13). The consequences for an encoded protein for each mutation are indicated below the nucleotide sequence, where each letter indicates a missense change, =indicates a silent change, * indicates a stop codon and ø indicates a splice site mutation. The position of Q479 to stop obtained in the screen is depicted as ▾ (See Table 1).

FIG. 5 depicts in the form of a cartoon the high throughput TILLING strategy applied to a plant such as Arabidopsis thaliana as demonstrated in Example 3.

### DESCRIPTION OF THE SPECIFIC EMBODIMENTS

Generally the subject invention relates to methods for finding multiple mutations in genes of known sequence by combining mutagenesis with methods for finding point mutations. The present invention provides a method for the creation and subsequent detection of mutations within a selected (desired) DNA region. The mutations created provide a range of allele types, including knockouts and missense mutations, which will be useful in a variety of gene function and interaction studies. This method is particularly useful for studies in organisms that do not have extensive genetic tools or genomic DNA sequence available.

In a specific embodiment of the present invention the genome of Arabidopsis is mutagenized to produce a plurality of different point mutations and screened with semi-automated nucleic acid amplification-based methods, i.e., PCR, within a gene region of interest. As an example, mutations in any gene contained in the genome of Arabidopsis can be screened by the methods of the present invention in as few as a set of approximately 5-10,000 reference plants. It is expected that most phenotypes can be scored in the F2 progeny of reference plants, and therefore functional analysis can be easily performed.

Although in the present disclosure TILLING has been specifically applied to Arabidopsis and Drosophila, the methods as described are of general use. Therefore, any organism that can be mutagenized can be TILLed, although plants are especially suitable. The general applicability of the methods of the present invention means that organisms lacking well-developed genetic tools can be TILLed. For example, but not by way of limitation, plants such as maize, alfalfa, barley, rice, soy beans, cotton, pine, melons, and other commercially important crop plants can be analyzed with the methods of the present invention. Additionally, model plant systems, such as the legume Medicago truncatula, can be examined using the methods of the present invention. In this context, seeds, pollen, germ cells and cells cultured from plants are suitable subjects for TILLING. In addition to plants, animals are also suitable subject for TILLING. Within this context, germ cells of animals such as nematodes, fruit flies, mice, chickens, turkeys, dogs, cats, cows, sheep, horses, pigs and other commercially important agricultural and companion animals can be analyzed with the methods of the instant invention.

TILLING is related to a method whereby chemically mutagenized Caenorhabditis elegans cultured in microtiter plates were screened by PCR for deletions (Liu et al., Genome Res. 9:859-867 (1999)). However, because this method requires screening of approximately 10⁶ genomes (about 100 times more than that required for TILLING) to obtain a knock-out mutation, it is not likely to be generally applicable. Instead, by screening with the methods provided herein for high frequency point mutations several advantages over previously disclosed reverse genetic methods are realized including, for example:

(1) Once genomic DNAs are prepared and arrayed, the process is almost fully automated. All subsequent steps, including, for example, detection of mutations, e.g., by PCR and dHPLC analysis, can typically be performed in, for example, microtiter plates, which can be handled robotically.

(2) Chemical mutagens, in particular, mutagens which result in primarily point mutations and short deletions, insertions, transversions, and or transitions (about 1 to about 5 nucleotides), such as ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7, 12 diinethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloro-ethyl)aminopropylamino] acridine dihydrochloride (ICR-170), formaldehyde, and the like and radiation provide reliable means to mutagenize the genome of the organism of interest, so that by choosing a suitable coding region, the probability of success in recovering a subtle phenotype such as those that are functionally hypo- or hypermorphic, or weak or weakened alleles and the like, knock-out, partially suppressive, or deleterious changes can be calculated in advance.

(3) Results are obtained on a plate-by-plate basis, so as soon as a desired mutation is found, screening can be terminated (McCallum et al., Plant Physiol. 123:439-442 (2000)).

(4) A range of useful missense mutations, not just knock-outs, are obtained. For instance, temperature-sensitive missense alleles, expected to be especially useful for gene interaction studies (Bowman et al., Plant Cell 1:37-52 (1989)), can be obtained.

(5) Because the optimal size of a PCR product useful for detection by, for example, dHPLC or gel electrophoresis following cleavage of an oligonucleotide strand at the position of a base mismatch, for example, by endonuclease digestion, is the size of a small gene, small targets that are likely to be missed by other methods, such as, high density transposon tagging strategies or large DNA detection strategies (Wisman et al., Plant MoL Biol. 37:989-999 (1998)); Bevan et al., Bioessays 21:110-120 (1999) each incorporated herein by reference) present no special problem for TILLING.

(6) Any gene can be targeted, whether essential or not, because mutations are detectable in both homozygotes, or heterozygotes.

(7) The methods of the present invention permits one to find mutations in a gene of interest in the absence of an assayable phenotype which may later be discerned, for example mutations that are present as a heterozygous mutation may not be detected in a heterozygote, but may be detectable when the identified mutations crossed and a homozygous individual is obtained.

(8) The use of gel electrophoresis, i.e., or other size separation method, in the instant invention permits the localization of mutations at or within a few base pairs of the mutation and thus permits the efficient identification of mutations in regions of interest.

(9) The use of EMS, nitrosoguanidine or 2-aminopurine, and the like, in certain embodiments permits knowledge of exactly what mutation has taken place because these mutagens result in a high (95% or greater) frequency of specific base substitutions (transitions or transversions such as GC to AT transitions). Thus upon identification of the location of the mutation, one can determine from the known sequence, what the identity of the mutated sequence is with a probability equal to the specificity of the base substitution of the mutagen.

Although radiation and chemical mutagenesis are reasonably efficient, reliable and well understood, they have not been widely used in reverse genetic methodology because point mutations, which are the primary induced lesions (Ashburner; Drosophila, A Laboratory Handbook, Cold Spring Harbor Press, Cold Spring Harbor (1990)), are difficult to detect. Typically, when practicing the methods of the present invention the concentration of the mutagen selected will be that which will induce a plurality of different mutations in the genome of the organism of interest.

Mutagenesis as used herein refers to methods for inducing a plurality of mutations in the DNA of a cell. The mutations typically usefull in the methods of the present invention are those which induce changes that alter or eliminate the function if the gene product (i.e., a nucleotide substitution, deletion, or insertion). The methods of the present invention are especially useful in detecting point mutations. “Point mutations” include single base transitions, base tranversions, insertions and deletions. Typically the mutagenesis comprises exposing a germ cell of an organism, a cell, or a seed with a chemical mutagen, i.e., but not limited to those listed above. The cells can also be treated with, for example, radiation, i.e., x-rays and gamma-radiation, which induce primarily larger lesions, ultra-violet light, and the like. In addition, a polynucleotide sequence encoding certain heterologous enzymes which can induce mutations, i.e., a phyophosphohydrolase, such as the bacterial MutT gene which makes AT to CG transversions, and the like, can be introduced into the cell or seed.

Appropriate mutation rates for mutagens will typically be in the range of about 1 mutation per 500 kilobase pairs (kbp) to about 1 mutation per 10 kbp. This rate of frequency of mutagenesis translates into the astounding level of 1 mutation in 5 to 25 genes. A rate that is three orders of magnitude higher than ever reported. The amount of mutagen necessary to achieve the desired mutation rate will be evident to one skilled in the art. Effective amounts of mutagen can be determined as generally described below. Briefly, a whole organism, germ cells, seed or cells, or the germ cells of an organism are collected and subjected to mutagenesis with varying amounts of mutagen. Germ cells are fertilized or self-fertilized and permitted to grow into organisms, or are permitted to proliferate. These cells or organisms which have the highest exposure to mutagen and still produce fertile offspring are used for later analysis and experiments. This population represents the highest mutation burden under which the organism (or cell) can function reproductively. Genomic DNA is obtained from the selected F2 individuals and pooled. The pooled DNA is subjected to TILLING as described below using primers designed for one, or typically multiple (usually up to at least 12) marker regions or other regions of interest. The mutation rate at a particular region or gene locus can thus be determined by dividing the number of mutations found in all regions by the total number of base pairs screened.

In a specific example provided herein EMS was used to induce primarily point mutations in Arabidopsis. Other agents are well known to the skilled artisan which provide similar results in various other organisms as provided hereinabove.

A sensitive, fast automated method is required for analyzing the large number of PCR-generated samples following mutagenesis. In one particular embodiment of the invention polymerase chain reaction (PCR) is used in the detection of mutations. PCR allows the skilled artisan to limit a search for mutations within specific regions of interest. Available methods for detection of mutations include, but are not limited to, single-stranded conformational polymorphism (SSCP) or heteroduplex analysis. Primers used for amplification are designed to specific regions of genes of interest. In one embodiment, primers were designed with melting temperatures of 60°-70° C., and final annealing temperatures of Tₘ−5° C. are chosen. Amplification products are denatured and reannealed under conditions permitting heteroduplexes to form. Such denaturation and annealing can be carried out in a separate step from the amplification or can be incorporated into the amplification protocol.

Further, heteroduplexes can be fragmented by chemical cleavage. Chemical cleavage can be carried out by, for example, hydroxylamine and osmium tetroxide to react with the mismatch in a DNA heteroduplex. subsequent treatment with piperidine cleaves the mismatched strand at the point of the mismatch. Mutations are detected by the separation of the fragments and the identification of fragments smaller than the untreated heteroduplex.

Heteroduplexes are also detectable by electrophoresis, for example by constant denaturant capillary electrophoresis (CDCE), or by denaturing high pressure liquid chromatography (dHPLC). Each of these methods has been used previously to identify naturally occurring polymorphisms consisting of single base changes in genes of interest (Gross et al., Hum. Genet. 105:72-78 (1999); Li-Sucholeiki et al., Electrophoresis 20:1224-1232 (1999) each incorporated herein by reference). The successes of these studies suggested that it was feasible to screen for single base changes in a population of mutations induced by an applied mutagen. In a specific embodiment of the present invention dHPLC was chosen for the screening of mutation because it combines automation, speed of analysis and high overall detection sensitivity for unknown single base changes in a commercially available instrument. Within another embodiment of the invention, endonuclease cleavage was used to identify mutations because it is a reliable and inexpensive point mutation discovery method that can be performed even more rapidly than dHPLC and in a robust manner.

Running time for dHPLC may limit throughput for the methods of the present invention, requiring about a week of screening for each mutation detected. However, it should be possible to increase throughput substantially by increasing the number of genomes represented in each pool. For example, the use of fluorescence detection rather than UV absorbance is expected to allow an order-of-magnitude increase in the number of genomes in a pool; smaller sample loads should minimize band broadening, allowing for better separation of heteroduplexes from homoduplexes. Fluorescence detection also may allow for multiplexing based on different fluorochromes, further increasing dHPLC throughput. Although fluorescence multiplexing is not yet commercially available for dHPLC, multiplexing has been performed by co-amplifying fragments that are differentially retained on the column but have similar melting temperatures.

Published studies utilizing DHPLC methodology have been limited to polymorphism discovery (Kuklin et al., Genetic Testing 1:201-206 (1997)). In such cases, it is important that mutations are not missed, and the nearly perfect detection rate reported for the method (Jones et al., Clin. Chem. 45:1133-1140 (1999)) is based on minimizing false negatives. However, in TILLING, false negatives only reduce the efficiency of mutation detection, and so the difference between 80% and 98% detection efficiency, though a serious drawback when looking for polymorphisms, requires that only 18% more plant genomes be TILLed. This means that one would be able to increase throughput by pooling more genomes and tolerating more false negatives. Thus far, false positives in screening Arabidopsis have not been encountered, although it is possible that larger genomes might require special measures to minimize PCR noise. If this turns out to be the case, then the low-cost precaution of reamplification and analysis by dHPLC can improve the results.

Heteroduplexes can be detected enzymatically, for example using an endonuclease that recognizes and cleaves at mismatches in a heteroduplex. Suitable endonucleases for use in the instant methods include resolvases, RNases, bacteriophage T4 endonuclease VII, bacteriophage T7, endonuclease I, Saccharomyces cerevisiae endonuclease X1, Saccharomyces cerevisiae endonuclease X2, Saccharomyces cerevisiae endonuclease X3, S1 nuclease, CEL I, P1 nuclease, or mung bean nuclease. Within one particular embodiment of the invention, the CEL I endonuclease (Oleykowski et al., Nucl. Acids Res. 26:4597-4602 (1998)) is used to cleave heteroduplex mismatches. CEL I, a plant-specific extracellular glycoprotein that belongs to the S1 nuclease family (Oleykowski et al., ibid.), has been shown to be suitable for genotyping applications because it preferentially cleaves mismatches of all types (Oleykowski et al., ibid.) and has been used to detect heterozygous polymorphisms in DNA pools (Kulinski et al., Biotechniques 29: 44-46 (2000)).

Within a particular embodiment of the invention, mutations are identified using a high-throughput TILLING method that utilizes an endonuclease to cleave heteroduplex mismatches. In the high throughput TILLING method described herein mutagenized DNA is first amplified using primers specific for a gene region of interest. The primers are preferably labeled with different independently detectable labels. This differential double-end labeling of amplification products allows for rapid visual confirmation, because mutations are detected on complementary strands, and so can be easily distinguished from amplification artifacts. The choice of labels useful in the methods of the present invention will be evident to the skilled artisan. Independent detection can be accomplished by, for example, using fluorochrome labels, i.e., fluorescein isothiocyanate (FITC), terachlorofluorescein, hexachlorofluoroscein, Cy3, Cy5, Texas Red, infrared dyes (IRDYE 700, IRDYE 770, IRDYE 800), or APC, and the like, that fluoresce at different wavelengths permitting clear identification of each label by its particular wavelength, or by selecting radioactive labels that are detectable using different filters. The amplification products are denatured and reannealed to permit the formation of heteroduplexes between the mutated and wild-type products.

Heteroduplex analysis is then carried out by cleaving the heteroduplexes with an endonuclease under conditions and for a time sufficient to permit endonuclease cleavage at mismatches between wild-type and mutant. Cleavage products are physically separated by, for example, gel electrophoresis or other means which exploits a change in size or mass. Slab gel electrophoresis is well suited for large-scale mutation detection. The two-dimensional readout facilitates the detection of rare events, such as mutations, because a new band will stand out above the wild-type background and can be easily spotted. The size of each new band is also obtained, an advantage over other methods based on detection of mismatches or conformational changes (Nataraj et al., Electrophoresis 20:1177-1185 (1999)), which do not indicate where in the molecule a mutation resides.

The separated cleavage fragments are differentially detected using methods suitable for the labels. Within one embodiment, IRDYE-labeled cleavage products separated in a polyacrylamide gel are detected by the measuring the absorbance at each wavelength characteristic of the label used, i.e., 700 or 800 nm, as the fragments pass through a detector. Images of the gel are obtained by for example, direct scanning or photography followed by scanning and the images are visually analyzed using graphic display software, such as Adobe PHOTOSHOP (Adobe, San Jose, Calif.), QUICKTIME (Apple Computer, Cupertino, Calif.), NETSCAPE NAVIGATOR (Netscape, Mountain View, Calif.), or the like. The images are analyzed with the aid of a standard commercial image processing program to identify the presence of change in fragment size indicating cleavage by the endonuclease at a mutation induced mismatch which give information on the presence of a mutation as well as its location. When used on pools of genomic DNA from individuals, mutations detected in a pool can be further investigated by screening the individual DNAs in the positive pools to identify the individual, e.g., plant, harboring the mutation. This rapid screening procedure determines the location of a mutation, or to within a few base pairs, for a PCR product up to 1 kb in size. Differential double-end labeling of amplification products allows for rapid visual confirmation, because mutations are detected on complementary strands, and so can be easily distinguished from amplification artifacts.

An additional important advantage of double end-labeling for detecting both cleavage products is avoidance of false positive bands. False positive bands which might result from the practice of the disclosed methods are of two types: those that appear in multiple lanes for a single detected label and those that appear in a single lane but in the same position for both detected labels. Within one embodiment, IRDYE detection is carried out by viewing the gel in each of two channels which detect a different infrared (IRDYE) label. Because it is highly unlikely that the same mutation will appear in two different individuals, it is assumed that certain homoduplex sites are especially sensitive to variability in endonuclease digestion, causing bands to appear in multiple lanes above the background pattern. Bands that appear for both labels are likely to be samples/pools in which mispriming leads to a large amount of double end-labeled product of a single size, with smaller products having a selective advantage over larger products during cycling. Such mispriming can lead to the detection of sporadic low molecular weight bands. In a particular embodiment, PCR product yield was determined to typically provide a low and inconsistent signal using both IR Dye 700 and IR Dye 800 dyes on opposing primers; however, consistent results have been obtained using a mixture of IRDye-labeled and unlabeled primers.

Within one contemplated improvement, detection and resolution of rare DNA molecules within mixtures are improved using capillary technology. For example, capillary electrophoresis has been successfully exploited for high throughput DNA sequencing (Kheterpal et al., Anal. Chem. 71 :31A-37A (1999)) and for rapid heteroduplex (CDCE) and SSCP detection applications (Larsen et al., Hum. Mutat. 13:318-327 (1999); Li-Sucholeild et al., Electrophoresis 20:1224-1232 (1999); Nataraj et al., Electrophoresis 20:1177-1185 (1999)). It is expected that dHPLC will also be accelerated by the development of capillary columns. Development of new separation or particular detection technology is outside the scope of the present invention, but should not be considered a limitation to the uses of the present methods.

Although TILLING minimizes the effort required to find mutations, ascertainment of a resulting phenotype requires additional characterization. Chemical mutagenesis introduces background mutations that can make phenotypic analysis uncertain, and multiple generations of outcrossing may be desirable. However, a rapid strategy is available if two independent severe lesions are found. Briefly, the two individuals can be crossed and their progeny typed. A phenotype attributable to the two non-complementing mutations will be found in every individual carrying both lesions, whereas non-complementing background mutations will sort independently.

About 25 missense mutations should be identified as a by-product of screening for two severe lesions. Because it is estimated that 5-10% of EMS-induced mutations are temperature-sensitive (Ashbumer, Drosophila, A Laboratory Handbook, Cold Spring Harbor Press, Cold Spring Harbor (1990)), the method of the present invention is likely to provide conditional mutants that can be used for epistasis and interaction analyses. Furthermore, by choosing evolutionarily conserved regions of proteins for TILLING, the probability of obtaining severe and conditional lesions is not only increased, but also mutations are provided in regions that are most useful for protein structure and function studies. The “Blocks” system, for example, is designed to find conserved regions amenable to the methods provided herein (Henikoffet al., Nucl Acids Res. 27:226-228 (1999)).

TILLING, as described herein, can be performed at a genomic scale to provide gene knockouts and conditional mutations for general study. For example, a collection of approximately 10,000 mutagenized reference M2 plants in an Arabidopsis race that is most suitable for TILLING has been partially established. The Columbia ecotype is a particularly suitable choice because it has been used for sequencing and EST analyses. Columbia erecta, a Columbia derivative that carries an induced erecta allele so that it has favorable compact growth characteristics (Yokoyama et al., Plant J 15:301-310 (1998)) has been used to establish the library. This line has been back-crossed to wild-type Columbia three times and self fertilized subsequent to EMS-mutagenesis, and so it is expected to be homozygous for about 90% of its genome. There should be only about 20 heterozygous mutations in the genome which could complicate the screening method described herein. However, even these heterozygotes can be eliminated by prescreening the unmutagenized parental genome.

Columbia erecta seeds are being mutagenized with EMS using the same protocol as described herein for plants. To avoid redundancy in mutations obtained each reference plant of the M2 generation can be grown from a separate M1 plant. It is important that DNA samples from different plants are nearly identical in concentration in order to maximize sensitivity to a mutation in any one sample plant.

In one embodiment of the present invention, two Arabidopsis chromomethylase genes (CMT2 and CMT3) related to CMT1 were selected. Primers were chosen based primarily on the probability of introducing a severe lesion. Mutations in the CMT2 and CMT3 genes were detected with denaturing HPLC (dHPLC), followed by sequencing to determine the mutation. Additionally, in another embodiment TILLING was used to examine functional mutations in a gene of known sequence in Drosophila. Within another embodiment, two Arabidopsis genes, hdal and Sir2B were selected and subjected to high-throughput TILLING. Primers were selected to flank the gene region of interest and to a specific Tm to facilitate amplification.

Other genes of known sequence can be chosen for TILLING using methods for analyzing the DNA sequence for regions which would have a high probability for mutation depending on the mutagen used. By assigning a score to defined regions of a target gene based on the likelihood of obtaining a desirable mutation, genes can be placed in a rank order. The ranks can be used both to pick regions of the selected gene for primers and to choose the order in which genes will be TILLed. Preliminary data with Arabidopsis suggests that approximately 5-10,000 reference plants will suffice for obtaining the desired mutations from just a single primer pair per gene that encompasses the most favorable region for TILLING. A computer program for choosing primers can output a list for oligonucleotide synthesis.

Plants are especially well suited to the methods of the present invention, because they can be self-fertilized and seeds can be easily stored. Although the only plant species with a nearly complete gene sequence database is Arabidopsis thaliana, which has been described herein as a specific example of high throughput TILLING, other crop plants can also benefit from TILLING. In particular, the same genes discovered in Arabidopsis can be studied in crop plants as listed above.

In a specific embodiment, genes in other plants which are the same or similar to those discovered in Arabidopsis can be selected. The identification of a similar gene can be accomplished, for example, using CODEHOP PCR primer design (Rose et al., Nucl. Acids Res. 26:1628-1635 (1998)). This is a PCR primer design method for amplification of distantly related sequences.

Typically, one using the methods of the present invention is interested in seeking mutations in a sequenced gene of interest. This greatly simplifies the task of identification, because all that is needed to find mutations is to perform a similarity search using the sequence of interest to query the database of mutant sequences constructed using TILLING. Therefore, each database entry can be, for example, a FASTA-formatted sequence, containing the mutation that was determined from the individual plant PCR products. Searching the TILLING database of mutant sequences (typically supplemented with a database providing a set of non-mutant controls) will return single entries for each mutation aligned with the query. The mutation itself can be easily pinpointed as (presumably) the only non-matching alignment pair. A user would search an amino acid sequence database to find an amino acid mutation or a nucleotide sequence database to identify a base mismatch. Each mutation can be confirmed by sequencing both strands. Confirmation of a heterozygous mutation by sequencing can be challenging even when both strands have been sequenced, however, computational methods exist for interpreting sequence trace data to identify heterozygous mutations.

The lack of a good reverse genetics methods may have been an impediment to organizing genomics of some plant species, and the methods of the present invention will likely spur genomics in neglected but important plants. Further, the generality of TILLING means that screening for mutations by these methods is applicable to animals. Currently, reverse genetic techniques in zebrafish are both labor and resource intensive and are not suitable for genome-scale analysis. Other potentially suitable systems include cultured cells, for example, mutagenized mouse embryonic stem cells, which can be stored frozen and implanted when needed to obtain mice for phenotypic analysis.

To illustrate the present invention the following non-limiting examples are provided herein below.

### EXAMPLE 1

### TILLING of Arabidopsis CMT1 and CMT2

The discovery and characterization of Arabidopsis CMT1, a DNA methyltransferase homologue with a chromodomain, termed a “chromomethylase” has been previously reported (Henikoff et al., Genetics 149:307-318 (1998)). It is thought that chromodomains target proteins to interact with specific chromatin determinants (Platero et al., EMBO J 14:3977-3986 (1995)); thus chromomethylases might be involved in epigenetic silencing phenomena by linking chromatin structure to DNA methylation. However, in several Arabidopsis thaliana races, CMT1 is found to be homozygous null. This non-essentiality of CMT1 could be explained as redundant function if other chromomethylases exist in Arabidopsis. To search for other chromomethylases, the CODEHOP PCR primer design method (Rose et al., Nucleic Acids Res. 26:1628-1635 (1998)) was employed and two different nucleic acid sequences evidently related to CMT1 from A. thaliana genomic DNA were isolated. Using these PCR products to probe an A. thaliana genomic library, two new chromomethylase genes were identified, CMT2 and CMT3. RT-PCR and isolation and sequencing of the full coding regions of CMT2 and CMT3 cDNAs revealed that their intron/exon boundaries are similar to those of CMT1 (FIG. 1). Quantitative RT-PCR expression studies showed that CMT2 and CMT3 are ubiquitously expressed at moderate levels, as might be expected for genes involved in silencing.

The biological functions of these new chromomethylases was not determinable by the identification of mutants using standard reverse genetics approaches. PCR screening of T-DNA lines available in 1998 (See, http://aims.cps.msu.edu/aims/) did not detect insertions in CMT2. Therefore, the antisense method was tried and 50 transgenic lines expressing CMT2 antisense RNA were obtained. However, Northern blot analysis of the antisense plants showed that the CMT2 sense transcript was still present in all 50 lines. Thus, two standard reverse genetic methods failed to affect expression of CMT2. The rate of failure of these standard methods is difficult to assess given that successes are eventually published, but failures are not. As such, no systematic survey that assesses the success rate of these widely used reverse genetic methods is apparently available.

In this example Arabidopsis thaliana has been mutagenized with ethyl methanesulfonate (EMS) and the chromomethylase 2 (CMT2) and chromomethylase 3 (CMT3) genes have been examined for mutations.

Cloning and Expression.

Conserved regions from CMT2 and CMT3 were amplified from A. thaliana genomic DNA using primer sets 5′-CATGGTTTGTGGAGGACCTCCNTGYCARGG-3′(SEQ ID NO: 1)+5′-TTGCA TCATTCCGAATCTACAYTGRTANYYCA-3′(SEQ ID NO: 2), and 5′-GTTAGAG AGTGTGCTAGACTTCARGGNTTYCC-3′(SEQ ID NO: 3)+5′-CAACAGGAACA GCAACAGCRTTNCCNAYYTG-3′(SEQ ID NO: 4), respectively, chosen using the CODEHOP primer designer and recommended cycling conditions (Rose et al., Nucleic Acids Res. 26:1628-1635 (1998)). The two PCR products were TA-cloned (Invitrogen) and sequenced. Two unique sequences related to CMT1 were identified and used to probe an A. thaliana genomic library (Clontech). The cDNA sample preparation and RT-PCR conditions used were previously described (Henikoff et al., Genetics 149:307-318 (1998)). Primer sets 5′-GTCTTTGGTGGGATGAAACTGT-3′(SEQ ID NO: 5) and 5′-CTTGAAGCTGAGGG TAAGTTGAAT-3′(SEQ ID NO: 6), CMT2; 5′-GTAAAAGCTT GCAGCATAACCAC-3′(SEQ ID NO: 7) and 5′-TAACTTTTTAGGGACTCCGAAGG-3′(SEQ ID NO: 8), (CMT3) and cyclophillin (Henikoffet al., Genetics 149:307-318 (1998)) were used for RT-PCR amplification.

EMS Mutagenesis Tissue Collection and DNA Extraction.

Seeds from A. thaliana ecotype No-0 were mutagenized with 20 mM EMS for 18 hours (Koornneef et al., Mutat. Res. 93:109-123 (1982)). Seeds from these M1 plants were collected in batch for the M2 generation.

Leaf samples from five M2 individuals were pooled prior to DNA extraction. To ensure that approximately equal amounts of tissue were collected from every individual, leaf samples were collected as punches using a #4 (9.5 mm diameter) cork borer and stored at −80° C. A modification of a quick DNA preparation protocol (Edwards et al., Nucleic Acids Res. 19:1349 (1991)) was used. DNAs from individual plants were prepared when a pool containing a mutation was identified.

PCR Amplification and dHPLC.

Samples for mutational screening and sequencing were generated in 20 μl reaction volumes containing approximately 1 ng pooled genomic DNA, 2.5 mM MgCl₂, 100 μM dNTPs, 0.2 μM of forward and reverse primers, 1X Pfu buffer and 2.5 U of Pfu polymerase (Stratagene). TOUCHDOWN PCR amplifications were performed as recommended by the manufacturer (Transgenomic Inc., San Jose, Calif.) (Kuklin et al., Genetic Testing 1:201-206 (1997)). Cycle sequencing protocols were used with ABI Model 373 sequencers.

Mutation detection was performed using the WAVE system (Transgenomic Inc., San Jose, Calif.). Following PCR amplification, the Pfu polymerase was inactivated while the DNA samples were heated and cooled to form heteroduplexes. For most fragments, the predicted WAVE (v.3.5) melting temperatures and separation gradients were used (Jones et al., Clin. Chem. 45:1133-1140 (1999)). For CMT2B and CMT3B, the software predicted two melting domains, and so the corresponding samples were analyzed at each of the predicted melting temperatures.

After EMS mutagenesis (Redei et al., in, Methods in Arabidopsis Research, pp. 16-82. World Scientific, Singapore, 1992; Feldmann et al., in, Arabidopsis, pp. 137-172. Cold Spring Harbor Press, Cold Spring Harbor, N.Y. (1994); Lightner et al., Methods on Molecular Biology, 82:91-104 (1998)) and self fertilization, DNA samples from several individual M2 plants were pooled, and pools were utilized as templates for PCR using primers that amplify a region of interest. To detect mutations, PCR reaction pools were heated and cooled to allow heteroduplexes to form between wild-type and mutant fragments, and denaturing HPLC chromatograms were obtained for each pool. Base changes were detectable as extra peaks owing to melting of duplex regions around mismatches and reduced retention on the heated reverse phase HPLC column. When a chromatographic alteration was detected, DNAs from individual plants were amplified and typed, and the PCR sample carrying the alteration was sequenced using an amplification primer.

To minimize screening time, a degree of sample pooling that would not compromise sensitivity was determined. Fragments derived from the CMT1 gene of 134 bp and 584 bp, previously determined to contain single base polymorphisms between the Landsberg (Ler) and Columbia (Col) ecotypes (Henikoff et al., Genetics 149:307-318 (1998)) were compared. Dilutions of Ler to Col ecotypes of 1:5, 1:10 and 1:20 were examined. The chromatograms demonstrated that the single base differences could be detected reliably using a UV detector in the 1:5 and 1:10 dilutions and in the 1:20 dilution with the shorter fragment (FIG. 3).

Both homozygotes and heterozygotes were expected to be present in the mutagenized population. Since homozygous mutations in some genes were expected be lethal or sterile, it was desirable to detect heterozygous mutations in these genes. If DNA aliquots from five individuals were present in a pool, then each mutation was diluted 1:5 in a homozygote and 1:10 in a heterozygote. Single base changes in both fragments at these dilutions were detectable. Therefore pooling samples from five individuals resulted in adequate sensitivity while producing a five-fold increase in detection efficiency.

Likelihood of Finding a Deleterious Mutation.

Three classes of mutations in protein-coding regions were expected to result from single base changes following chemical mutagenesis. First, nonsense mutations result from single base changes that convert an amino acid codon into a stop codon. Second, missense mutations result when single base changes alter the amino acid encoded by a particular codon; these can be further categorized as those resulting in conservative and nonconservative substitutions. Third, silent mutations result when a single base change to a codon does not alter the encoded amino acid. These changes are usually, but not exclusively, the result of mutations that alter the third base of a codon. Because nonsense and missense mutations that result in nonconservative substitutions are most likely to result in deleterious mutations, it is important to know the expected frequency of each class of mutation.

In Arabidopsis, EMS produces primarily C to T changes resulting in C/G to T/A transition mutations. For example, an examination of the LEAFY EMS-generated alleles (http://www.salk.edu/LABS/pbio-w/Ifyseq.html) reveals that 20/23 are CIG to T/A, 2/23 are C/G to A/T, and 1/23 is A/T to T/A. Assuming that all changes are C/G to T/A transitions and using the standard Arabidopsis codon usage table (http://www.kazusa. or.jp/codon/cgi-bin/ showcodon.cgi?species=Arabidopsis+thaliana+[gbpln]), an overall 5% of mutations were calculated to introduce a stop codon, 65% were calculated to be missense mutations, and 30% to be silent changes. These frequencies are calculated explicitly for each potential amplicon (e.g., FIG. 4), allowing the frequency of a deleterious allele to be maximized in the selection of the PCR fragment. For example, some fragments have a higher than expected concentration of the four codons (TGG, CAG, CAA and CGA) that can undergo a C/G to T/A mutation to produce a stop codon. Furthermore, by choosing coding regions that are evolutionarily highly conserved, the likelihood of recovering missense mutations with detrimental effects on gene function can be maximized. In addition to these coding region mutations, transition mutations in splice junctions are deleterious, and so for every intron in a chosen region there are at least two positions at which C/G to T/A mutations lead to loss of gene product. It can be calculated that overall 1% of the mutations in coding regions will be disruptions of splice junctions.

Detection of Mutations in CMT2 and CMT3.

A screen for mutations in a cell of interest demonstrated as an example the methods of the present invention analysis of the CMT2 and CMT3 using 835 M2 plants. Seven different PCR fragments ranging in size from 345-970 bp were examined, for a total of approximately 2 Mb of DNA sequence screened by dHPLC. Thirteen chromatographic alterations were detected and confirmed to be mutations by amplification of multiple samples (Table 1); no PCR errors were found. Analysis of the isolated DNA demonstrated some fragments have a higher than expected concentration of the four codons (TGG, CAG, CAA and CGA) that can undergo a CIG to T/A mutation to produce a stop codon. Furthermore, by choosing coding regions that are evolutionarily highly conserved, the likelihood of recovering missense mutations with detrimental effects on gene function can be maximized. In addition to these coding region mutations, transition mutations in splice junctions are deleterious, and so for every intron in a chosen region there are at least two positions at which C/G to T/A mutations lead to loss of gene product. It can be calculated that overall 1% of the mutations in coding regions will be disruptions of splice junctions.

Detection of Mutations in CMT2 and CMT3.

A screen for mutations in a cell of interest demonstrated as an example the methods of the present invention analysis of the CMT2 and CMT3 using 835 M2 plants. Seven different PCR fragments ranging in size from 345-970 bp were examined, for a total of approximately 2 Mb of DNA sequence screened by dHPLC. Thirteen chromatographic alterations were detected and confirmed to be mutations by amplification of multiple samples (Table 1); no PCR errors were found. Analysis of the isolated DNA demonstrated an error rate of <10⁻⁶. All detected mutations were base transitions in either homozygotes or heterozygotes, as expected for EMS-mutagenized M2 plants.

In CMT2, one mutation resulted in an Asp to Asn amino acid change and another was detected within an intron. Two different changes in nucleotide sequence were identified in CMT3. One mutation changed a Glu codon to Lys, and the other changed a CAG Glu codon to a TAG stop codon. The stop codon resulted in truncation of CMT3 (FIG. 4), which lacked four conserved blocks that are known to be crucial for enzymatic function (Posfai et al., Nucleic Acids Res. 17:2421-2435 (1989)). Transition mutations were discovered in nine other plants, but each of these is identical to one of the mutations described above. This finding can be attributed to sampling from the same mutagenized zygotes: seeds for the M2 generation were sampled at random from a pool of seeds produced by M1 plants, and it appeared that perhaps half of the M2 plants that were screened were redundant.

Thus, it was estimated that by screening 4 kb within about 400 different zygotes, 4 independent mutations were detected, at least one of which is a truncation that knocks out CMT3. The identification of 3 individual plants that were homozygous for the CMT3 knock-out, as described below, in the No-0 line, which is homozygous null for CMT1 (Henikoffet al., Genetics 149:307-318 (998)) demonstrated that loss of function for at least two chromomethylases was compatible with viability.

TABLE 1
Screen for CMT2 and CMT3 mutations.

| Frag- | Size | # | Mu- | | | Protein |
|---|---|---|---|---|---|---|
| ment | (bp) | Screened | tations | Allele | Zygosity | change |
| CMT2A | 970 | 375 | No | | | |
| CMT2B | 528 | 460 | No | | | |
| CMT2C | 422 | 375 | No | | | |
| CMT2D | 589 | 835 | No | | | |
| CMT2E | 500 | 835 | G to A | CMT2-1 | homozygous | D1214 |
| | | | G to A | CMT2-2 | heterozygous | to N |
| | | | | | | intronic |
| CMT3A | 345 | 835 | G to A | CMT3-1 | homozygous | E352 |
| | | | | | homozygous | to K |
| | | | | | homozygous | |
| | | | | | heterozygous | |
| | | | | | n.d. | |
| | | | | | n.d. | |
| CMT3B | 567 | 835 | C to T | CMT3-2 | homozygous | Q479 |
| | | | | | homozygous | to stop |
| | | | | | homozygous | |
| | | | | | n.d. | |
| | | | | | n.d. | |
| CMT3C | 550 | 460 | No | | | |

n.d. = not determined

### EXAMPLE 2

### TILLING the Drosophila Genome

This example provides a method for the examination of functional mutations in a gene of known sequence in fruit flies.

Male flies will be fed EMS to induce point mutation in the genome. The males will be crossed enmass to Balancer⁺ (Bal⁺) females (where the balancer chromosome is used to suppress recombination and maintain heterozygous lines). As flies emerge M1 Bal/*×Bal/* (wherein * means mutagenized chromosome) matings are set up, removing the parent flies after a sufficient egg-laying period. The number of males and females collected in each vial from the mating will depend on the sensitivity of the method of detection and the dose of mutagen used. It is expected that about 10 females and 10 males would result in about 10 * genomes represented among the resulting brood.

Matings for the M2 will be carried out selecting about 20 aged non-Bal (*/*) and allowing egg laying. The vial will contain a sampling of mutagenized genomes and Bal chromosomes. Flies in the vial will be allowed to develop at low temperature (about 14° C.) to hold the M3 generation as long as possible.

DNA will be prepared from the */* M2 parents in 8×12 arrays. It is expected that each independently mutagenized chromosome might be as rare as 1/40, and would presumably be missed. However, the representation of the average chromosome should be 1/10. Therefore, about half of the mutations will be detected on average, depending on the fragment size for screening.

Screening of a gene of interest will be carried out by PCR and dHPLC of replicate samples. As soon as possible upon detection of a positive chromatogram, single males from the M3 vial will be crossed to fresh Bal/⁺ females to prevent losing the identified mutation. From each resulting M4 vial DNA will be prepared from about 20 non-Bal (*1/⁺ and *2/⁺) adults for PCR and dHPLC analysis. From a positive M3 vial, several single Bal/*×Bal/marker flies will be crossed. Half of these crosses should yield */marker adults with positive chromatograms. Sequence quality DNA will be prepared from a positive sample and the sequence of the mutation determined. Stocks will be set up, and outcrosses performed and complementation crosses can be carried out if needed.

### EXAMPLE 3

### High-Throughput TILLING

This Example describes a high-throughput TILLING method which was used to analyze the Arabidopsis hda1 gene. As described in more detail below, in the high-throughput TILLING method, a region of interest was amplified using a “left” primer labeled with a first label and a “right” primer labeled with a second label and wherein the labels are independently detectable. Heteroduplexes of the amplified and labeled DNA was nicked at a mismatch with the endonuclease CEL I. The use of label at both ends of the amplification products permitted identification of mutations and sequencing at single base resolution from no farther than the middle of any fragment, thus allowing for larger segments of a gene to be analyzed. In addition, using two different labels that permit the independent detection of both labels in a non-overlapping manner on the same gel simplified comparisons and helped to identify artifacts, which appeared with both labels. Within this example, because of low efficiency priming of IR dye-labeled (IRDYE) oligonucleotides, unlabeled primers were added in excess to permit efficient priming on template and early in the reaction thus providing higher concentrations of product for IR dye-labeled primers to anneal during later cycles. The amplification products were digested with CEL I and the cleavage products were subjected to denaturing electrophoresis through an acrylamide gel. Labeled cleavage products were identified by detecting each labeled DNA strand and images of the gel were captured and analyzed by scanning and image software.

Determination of Pool Size

Initial experiments were performed by using 5-fold pools of individual mutagenized plants, which appeared to be the practical limit of detection by dHPLC for fragments in the 5-600 bp range (Example 1 and McCallum et al., Nature Biotechnol. 18:455457 (2000)). By screening for mutations in the Arabidopsis Sir2B gene using both dHPLC method (Example 1) and the method described in more detail below, detection levels were directly compared. High-throughput TILLING on 5-fold pooled samples for the Sir2B gene was compared with 5-fold pooled samples that were carefully screened using dHPLC, with products confirmed by DNA sequencing. Six confirmed mutations, all heterozygous G/C to A/T transitions, were detected by both methods; four mutations were detected using dHPLC and five mutations were detected by the high-throughput TILLING method. An increase in pooling to 8-fold, resulted in similarly high detection levels without false positives: in one test. A screening of 4-fold pools found only the same 7 mutations discovered in 8-fold pools of the same DNAs. Therefore, an 8-fold pooling scheme was found to be preferable.

Mutagenesis.

Starting with a single plant of Arabidopsis thaliana, ecotype Columbia homozygous for an erecta mutation (Torii et al., Plant Cell 8: 735-746 (1996)), seeds were collected and mutagenized in batches at 20 mM, 25 mM or 30 mM EMS as described in Example 1 and in McCallum et al. (Nature Biotechnol. 18: 455-457 (2000)). M1 plants were allowed to grow in trays and to self-fertilize. The resulting seeds were sown in pots for the M2 generation, where each M2 was derived from a different M1 plant. Genomic DNA from each M2 plant was prepared from 0.2 g of leaf and/or stem tissue using the BIO101 FASTDNA system (Qbiogene, Carlsbad, Calif.) following the manufacturer's instructions. Concentrations of the DNA preparations were estimated by visualization on 1% agarose electrophoretic gels and were equalized prior to dilution (in 10 mM Tris pH 8.0, 1 mM EDTA) and pooling. The genomic DNA samples from each individual plant were pooled at either 5-fold or 8-fold representing either 5- or 8-individual plants per well, and the pools were arrayed on microtiter plates.

Amplification of Genomic DNA Pools.

The genomic DNA pools were subjected to hda1 gene-specific amplification using polymerase chain reaction (PCR) using primers designed with melting temperatures of 60°-70° C., and final annealing temperatures of Tₘ₋5° C. were chosen. Briefly, each PCR amplification reaction was performed in 10 μL volumes using EXTAQ polymerase (PanVera Corporation, Madison, Wis.) using the manufacturer's protocol with the exception that only half the manufacturer's recommended concentration of buffer was used, and MgCl₂ concentration was increased to 2 mM. Primers (forward primer: 5′ GGTAATGGATACTGGCGGCAATTCG 3′ (SEQ ID NO: 9), reverse primer: 5′ ACCACCCAAGAGCAGTAGGGGAACA 3′; SEQ ID NO: 10) were obtained from MWG Biotech (MWG Biotech Inc., High Point, N.C.). The forward primer was labeled with the infrared detectable label IRDYE 700 (IRDYE 700 (LI-COR Inc., Lincoln, Nebr.), molecular formula: C₅₂H₆₇N₄O₅PS) and the reverse primer was labeled with the infrared red detectable label IRDYE 800 (IRDYE 800 (I-COR Inc.), molecular formula: C₅₉H₇₅N₄O₆PS). The primers were mixed in a ratio of 3:2 labeled to unlabeled primer (IRDYE 700-labeled primer) and 4:1 labeled to unlabeled primer (IRDYE 800-labeled primer), for final primer concentrations of 0.2 μM.

The reaction mixtures were subjected to amplification cycles in a MWG Biotech 96-well cycler (MWG Biotech Inc.) as follows: 1) 95° C. for 2 min; 2) 8 cycles of TOUCHDOWN PCR: 94° C. for 20 sec (denaturation), Tₘ+3° c to Tₘ−4° C. decrementing 1° C. per cycle (annealing), 72° C. for 45 sec to 1 min (extension for 600 to 1000 bp products); 3) 45 cycles of: 94° C. for 20 sec (denaturation), Tₘ−5° C. (annealing), 72° C. for 45 sec to 1 min; 4) 72° C. for 5 min; 5) 99° C. for 10 min (inactivation); 6) 70 cycles of 20 sec at 70° C. to 49° C., decrementing 0.3 C per cycle (reannealing).

Production of CEL I Enzyme.

The CEL I enzyme, an endonuclease that preferentially cleaves mismatches in heteroduplexes between wildtype and mutant, was purified from 30 kg of celery essentially as described by Oleykowski et aL (Nucleic Acids Res. 26: 4597-4602 (1998); which is incorporated herein by reference in its entirety), except that POROS HQ (an anion exchangers, surface coated with quaternized polyethyleneimine, PerSeptive Technology, Foster City, Calif.) rather than Mono Q (beaded hydrophilic resin where the base matrix is substituted with quarternary amine groups (Amersham Pharmacia Biotech, Piscataway, N.J.) was used, and the Whatman P11 1(P 11 is a bifunctional cation exchange cellulose, Whatman, Ann Arbor, Mich.) and S columns (Amersham Pharmacia Biotech) were omitted. The specific activity was 1×10⁶ units per ml, where a unit is defined as the amount of CEL I required to digest 50% of 200 ng of a 500 bp DNA fragment that has a single mismatch in 50% of the duplexes.

CEL I Cleavage Reactions.

Amplification products were incubated with CEL I and cleavage products were then electrophoresed using an automated sequencing gel apparatus, and gel images are analyzed with the aid of a standard commercial image processing program. Briefly, 10 μl of each amplification product was mixed with 20 μl of CEL I buffer (10 mM Hepes pH 7.5, 10 mM MgSO₄, 0.002% Triton-X-100, 20 ng/ml of bovine serum albumin) and 1/1000 dilution of CEL I(50 units/μL) on ice. The reactions were incubated at 45° C. for 15 minutes. Reactions were stopped by addition of 5 μl 0.15 M EDTA (pH 8). The entire volumes of the reaction mixtures were transferred into the wells of a SEPHADEX G50 (dextran gel filtration matrix, Amersham Pharmacia Biotech) spin plate.

The Sephadex G50 spin plates were prepared according to the manufacturer's recommendation. Briefly, G50-150 (medium) powder was distributed evenly into a 96-hole metal plate. A 96-well membrane plate was fitted on top of the metal plate and the two plates were inverted and tapped to fill the wells with powder. Approximately 300 ml of water was added to the top of each well to hydrate the powder. The plates were covered and allowed to sit for at least 1 hour at room temperature. A blue alignment frame adapter (Millipore, Bedford, Mass.) and a waste plate were attached to the bottom of the spin plate. Plates were stored at 4° C. in a sealed bag to prevent drying.

MWG 96-well catch plates (MWG Biotech Inc.) were prepared by transferring about 1 to about 1.5 μl formamide load solution (1 mM EDTA pH 8 and 200 μg/ml bromphenol blue in deionized formamide) into each well of a fresh MWG 96-well catch plates (labeled and oriented).

After the CEL I reactions were stopped, loaded onto the spin plates and prior to spinning, the waste plate on the spin plate was replaced with a catch plate. The plates were spun for 2 minutes at 1760 rpm uncovered. The volume of each reaction was reduced to approximately 1.5 μλ, and the DNA denatured by incubating the plates at 96° C. uncovered for about 30 to 40 minutes. After the reaction volume had been reduced, the plates were transferred to ice until ready for loading.

The reactions were subjected to denaturing gel electrophoresis by first transferring the reactions to a membrane comb using a comb-loading robot and the COMBLOAD program supplied by the manufacturer (MWG Biotech). An IRDYE 800-labeled 50-700 bp molecular weight marker mix (LI-COR Inc.) was applied to outside teeth. Following the prerun-focusing step on a LI-COR Global IR² gel scanner (LI-COR Inc.), the comb containing the CEL I-treated amplification products was inserted into a well on top of a 6.5% acrylamide gel, electrophoresed for 1 min and removed. Electrophoresis was continued for 4 hours at 1500 V, 40 W, 40 mA limits at 50° C.

Detection of CEL I Cleavage Products.

The DNAs were detected in two separate channels by a LI-COR scanner as generally described by Middendorf et al. (Electrophoresis 13: 487494 (1992); incorporated by reference herein in its entirely). As described in more detail below, this method was sufficiently sensitive to detect the approximately 100 atamole of cleavage product generated by CEL I in an 8-fold pool, or one in 16 genomes for a heterozygous mutation. The opposed PCR primers carried different dye labels. As there is no detectable overlap between the IRDYE 700 and IRDYE 800 dye labels, images were examined directly for the presence of novel bands in either channel, 700 nm or 800 nm wavelengths of excitation. The image files were visually analyzed using a graphics display software program such as Adobe Photoshop (Adobe, Inc., San Jose, Calif.). The images resulting from the gel scans showed a sequence-specific pattern of background bands resulting from endonucleolytic cleavages common to all 96 lanes. By superimposing images representing both channels and switching between them, lanes containing a novel band in one channel and a corresponding novel band in the other channel were identified. The sum of the two band sizes was equal to the full-length product visible at the top of the image. This visual assay was aided by the approximate proportionality of the migration distance to molecular weight, so that a band in one channel was nearly the same distance from the leading edge as the corresponding band in the other channel was from the full-length product. Image manipulation tools, rulers and guides were used for the determination of migration distances and lane numbers for the two bands.

Using the 8-fold pooling scheme, a total of about 750 kb of sequence has been interrogated for point mutations on a single gel. Differential double-end labeling of amplification products permitted rapid visual confirmation, because mutations were detected on complementary strands, and so were easily distinguished from amplification artifacts. 7 mutations were obtained and identified by CEL I digestion. Each band (CEL I product) has a corresponding band (same lane) in the other detection channel which results from digestion of the DNA product on the opposite complementary DNA strand. The size of the corresponding band is: length of the full length PCR product minus the size of the first band. The presence of these two bands in the same lane on the gel (each detected by detecting a different label) whose sizes add to the size of the original DNA PCR product confirms the location of a mismatch from both ends of the PCR product.

Identification of Plant Carrying a Mutation Identified by High-Throughput Tilling.

Upon detection of a mutation in a pool, the individual DNA samples were similarly screened to identify the plant carrying the mutation. This rapid screening procedure determined the location of a mutation or within a few base pairs for PCR products up to 1 kb in size. Moreover, the combination of Cell and EMS induced mutagenesis permits one to simultaneously identify and localize the mutation. Because EMS causes specific transition mutations, the use of this method permits one to determine the sequence of the mutation upon examination of the reference sequence, the wild-type sequence of the mutation and this is true of other mutagens as well.

Briefly, the individual DNA samples comprising the pool containing an identified mutation were screened. Individual DNA samples were arrayed in an 8×8 grid on microtiter plates, such that each pool corresponded to a row of individuals; thus each column of the pool plate corresponded to a column of rows in the 8×8 grid. The DNA was transferred from the row corresponding to the positive pool into a column of a fresh microtiter plate, so that 12 mutations per plate are screened as individuals. In order to detect homozygotes as heteroduplexes, the individual samples were mixed with an equal amount of wild-type DNA. From this point on, screening to detect 12 individual mutations was identical to screening of pools described above, including amplification, CEL I digestion, gel electrophoresis, and file transfer, Image and molecular weight analyses. This screen resulted in the identification of the plant in which a point mutation had occurred and an estimated location within a few base pairs of the lesion as well as confirming the original detection event in the 8-fold pool screen. Each mutation found in the arrayed plates of individual genomic DNA was re-confirmed by DNA sequencing.

This method provides for screening and identifying the plants which harbor detected mutations. But the method also provides, based on the size of the DNA fragments obtained on separation, i.e., gel electrophoresis, the location of the mutation. With this information, the skilled artisan can determine which of the detected mutations lies in a region of interest, i.e., mutations in regions that are most likely to have a biologically functional effect, and eliminate those in other regions, such as intron and regions of low protein sequence conservation. The elimination of the need to examine all mutations in these regions saves time and allows the artisan to focus efforts on the regions of biological interest. Additionally, with the precise location of a mutation and the use of a specific mutagen, the sequence, or identity, of the mutation can be known. For instance, greater than 99% of the changes in nucleotides made by EMS are GC to AT transitions. Thus the changes at a particular site of a G or a C is greater than 99% likely to be the corresponding transition to T or A.

Using this two-step strategy, as much as approximately 750 kb of individual genomic sequences per gel (1 kb x 8 plant DNAs×96 lanes) have been interrogated, and more than 75 mutations in Arabidopsis chromatin genes have been identified (Http://Ag.Arizona.Edu/chromatin/atgenes.html). For the most heavily mutagenized plants that were screened, which displayed 30% embryo lethality after the first round of selfing, approximately 10 point mutations were estimated per 8-fold pool plate (representing 768 plants) per gel for 1 kb fragments. This corresponds to approximately 1000 EMS-induced mutations per Arabidopsis genome.

Although the foregoing invention has been described in some detail by way of illustration and example for purposes of clarity of understanding, it will be obvious that certain changes and modifications may be practiced within the scope of the appended claims. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted.

What is claimed is:

1. A method for identifying functional mutations in a gene of known sequence comprising:
reating an organism or cell with a mutagen which primarily induces point mutations in the genomic DNA of the organism or cell;
isolating genomic DNA from the mutagenized organism or cell;
implifying a segment of the gene of known sequence to produce amplification products
denaturing and renaturing the amplification products to produce heteroduplexes
cleaving the heteroduplexes with an enzyme capable cleaving DNA at a mismatch in the heteroduplexes to produce cleavage products.
dentifying mutations in the unmutagenized organism or cell of the gene as compared to the sequence of the gene in the mutagenized organism or cell by detecting heteroduplex cleavage products.

2. The method according to claim 1, wherein the heteroduplexes are cleaved using an enzyme.

3. The method according to claim 2, enzyme is an endonuclease.

4. The method according to claim 3, wherein the endonuclease is bacteriophage T4 endonuclease VII, bacteriophage T7, endonuclease I, Saccharomyces cerevisiae endonuclease X1, Saccharomyces cerevisiae endonuclease X2, Saccharomyces cerevisiae endonuclease X3, S1 nuclease, CEL I, P1 nuclease, or mung bean nuclease.

5. The method according to claim 1, wherein the amplification products are cleaved with a chemical agent or radiation.

6. The method according to claim 5, wherein the chemical mutagen is ethyl methanesulfonate (EMS), methylmethane sulfonate (MMS), N-ethyl-N-nitrosourea (ENU), triethylmelamine (TEM), a diepoxyalkane, 2-methoxy-6-chloro-9[3-(ethyl-2-chloro-ethyl)aminopropylamino] acridine dihydrochloride (ICR-170), nitrosoguanidine, N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), 7, 12 dimethylbenz(a)anthracene (DMBA), ethylene oxide, hexamethylphosphoramide, bisulfan, 2-aminoguanidine, or formaldehyde.

7. The method according to claim 6, wherein the diepoxyalkane is diepoxyoctane (DEO), or diepoxybutane (BEB).

8. The method according to claim 6, wherein the chemical mutagen is EMS, nitrosoguanidine, or 2-aminopurine.

9. The method according to claim 8, wherein the mutagen is EMS and the endonuclease is Cell and wherein the mutation is identified simultaneously with the sequence of the mutation.

10. The method according to claim 5, wherein the radiation is x-rays, gamma-radiation, or ultra-violet light.

11. The method according to claim 1, wherein the organism is a plant or animal.

12. The method according to claim 11, wherein the plant is Arabidopsis, a legume, maize, alfalfa, wheat, barley, rice, soy beans, cotton, melon, tomato, or pine.

13. The method according to claim 11, wherein the animal is Drosophila, zebrafish or Caenorhabditis.

14. The method according to claim 5, wherein the cleavage products are separated by a denaturing size separation method followed by detection of the heteroduplex cleavage products.

15. The method according to claim 14, wherein the size separation method is gradient gel electrophoresis or capillary electrophoresis.

16. The method according to claim 1, wherein the mutation detected comprises a base transition or base transversion.

17. The method according to claim 1, wherein the mutation causes a missense or nonsense mutation.

18. The method according to claim 1, wherein the step of amplifying is carried out using a first primer specific for the 5′ end of the cleavage product, wherein the first primer is labeled by a first label and the second primer is labeled by a second label.

19. The method according to claim 2, wherein the heteroduplex cleavage products are detected by detecting the first and second labels.

20. The method according to claim 18, wherein the labels are chromophores, fluorophores or radiolabels.

21. The method according to claim 18, wherein the label pair is selected from the group consisting of fluorescein isothiocyanate (FITC), tetrachlorofluorescein, hexachlorofluoroscein, rhodamine, Cy3, Cy5, Texas Red, an infrared dye, and APC.

22. A method for identifying functional mutations in a gene of known sequence comprising:
treating an organism or cell with a mutagen which primarily induces point mutations in the DNA of the organism or cell;
isolating genomic DNA from the mutagenized organism or cells;
amplifying a segment of the gene of known sequence to produce an amplification product;
denaturing and reannealing the amplification product to produce a heteroduplex; and
identifying a point mutation in the gene segment as compared to the sequence of the gene in the parent organism or cell.

23. The method according to claim 22, wherein the mutagen is a chemical or radiation.

24. The method according to claim 23, wherein the chemical mutagen is ethyl methanesulfonate (EMS), methylmethane sulfonate (MMS), N-ethyl-N-nitrosourea (ENU), triethylmelamine (TEM), a diepoxyalkane, 2-methoxy-6-chloro-9[3-(ethyl-2-chloro-ethyl)aminopropylamino] acridine dihydrochloride (ICR-170), nitrosoguanidine, N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), 7, 12 dimethylbenz(a)anthracene (DMBA), ethylene oxide, hexamethylphosphoramide, bisulfan, 2-aminoguanidine, or formaldehyde.

25. The method according to claim 24, wherein the chemical mutagen is diepoxyalkane is diepoxyoctane (DEO), or diepoxybutane (BEB).

26. The method according to claim 24, wherein the chemical mutagen is EMS, nitrosoguanidine, or 2-aminopurine.

27. The method according to claim 22, wherein the radiation is x-rays, gamma-radiation, or ultra-violet light

28. The method according to claim 22, wherein the organism is a plant or animal.

29. The method according to claim 27, wherein the plant is Arabidopsis, a legume, maize, alfalfa, wheat, barley, rice, soy beans, cotton, melon, tomato, or pine.

30. The method according to claim 27, wherein the animal is Drosophila, zebrafish or Caenorhabditis.

31. The method according to claim 22, wherein a mutation in the gene is detected by single-stranded conformational polymorphism or heteroduplex analysis.

32. The method according to claim 31, wherein the heteroduplex analysis is denaturing high pressure liquid chromatography, followed by sequence analysis.

33. The method according to claim 31, wherein the heteroduplex analysis comprises fragmenting the heteroduplexes and detecting the presence of a mismatch in the heteroduplex by a change in the size of the fragments produced.

34. The method according to claim 33, wherein the heteroduplexes are fragmented using an enzyme.

35. The method according to claim 34, enzyme is an endonuclease.

36. The method according to claim 35, wherein the endonuclease is bacteriophage T4 endonuclease VII, bacteriophage T7, endonuclease I, Saccharomyces cerevisiae endonuclease X1, Saccharomyces cerevisiae endonuclease X2, Saccharomyces cerevisiae endonuclease X3, S1 nuclease, CEL I, P1 nuclease, or mung bean nuclease.

37. The method according to claim 33, wherein the amplification products are fragmented with a chemical agent or radiation.

38. The method according to claim 33, wherein fragments are separated by denaturing gradient gel electrophoresis or denaturant capillary electrophoresis.

39. The method according to claim 31, wherein the mutation detected comprises a base transition or base transversion.

40. The method according to claim 31, wherein the mutation causes a missense or nonsense mutation.

41. The method according to claim 31, wherein the step of amplifying is carried out using a first primer specific for the 5′ end of the gene segment and a second primer specific for the 3′ end of the gene segment.

42. The method according to claim 41, wherein each primer in the primer pair is labeled on with a different label.

43. The method according to claim 42, wherein the labels are chromophores, fluorophores or radiolabels.

44. The method according to claim 43, wherein the label pair is selected from the group consisting of fluorescein isothiocyanate (FITC), tetrachlorofluorescein, hexachlorofluoroscein, Cy3, Cy5, Texas Red, an infrared dye, and APC.
